(19) United States
(12) Patent Application Publication
(10) Pub. No.: US 2025/0009404 A1
(43) Pub. Date: Jan. 9, 2025

(54) MULTI-ELECTRODE DEVICES, SYSTEMS, AND METHODS FOR MEDICAL PROCEDURES

(71) Applicant: BOSTON SCIENTIFIC SCIMED, INC., MAPLE GROVE, MN (US)

(72) Inventors: Laura Emily Sabban, Plainville, MA (US); Serena Scott, Worcester, MA (US); Timothy A. Ostroot, Cokato, MN (US)

(73) Assignee: BOSTON SCIENTIFIC SCIMED, INC., MAPLE GROVE, MN (US)

(21) Appl. No.: 18/762,821

(22) Filed: Jul. 3, 2024

Related U.S. Application Data
(60) Provisional application No. 63/525,004, filed on Jul. 5, 2023.

(51) Int. Cl. A61B 18/00

(52) U.S. Cl. CPC A61B 18/00; A61B 2018/00077; A61B 2018/00083; A61B 2018/00178; A61B 2018/00184; A61B 2018/00214; A61B 2018/00273; A61B 2018/00577; A61B 2018/00613

(57) ABSTRACT

A multi-electrode energy-delivering assembly configured to be delivered in a generally elongate delivery configuration. The multi-electrode energy-delivering assembly includes one or more spaced apart electrodes of a first polarity. The electrodes may be spaced apart axially and/or may be expanded apart from one another upon deployment. In some aspects, the electrodes may be electrically activated independently of one another.

### CROSS-REFERENCE TO RELATED APPLICATIONS

This application claims the benefit of priority under 35 U.S.C. § 119 to U.S. Provisional Application No. 63/525,004, filed Jul. 5, 2023, the entire disclosure of which is hereby incorporated by reference herein for all purposes.

### FIELD

The present disclosure relates generally to the field of medical devices, assemblies, systems, and methods used in applying energy to a patient such as for therapeutic purposes. More particularly, the present disclosure relates to the field of medical devices, assemblies, systems, and methods for applying electrical energy, such as therapeutic electrical pulses, to a patient. Even more particularly, the present disclosure relates to various devices, assemblies, systems, and methods for electroporation treatment. And, even more particularly, the present disclosure relates to multi-electrode devices, assemblies, and systems applying energy, such as electrical energy such as for electroporation, and associated methods.

### BACKGROUND

Various devices, assemblies, systems, and methods exist for energy-based medical treatment and/or therapeutic protocols. For instance, various focal therapy devices are configured to apply energy to debulk target tissue or to eliminate malignant cells. Various technologies for such therapy rely on thermal effects, such as radiofrequency (“RF”) heating, microwave heating, cryoablation, high intensity focused ultrasound (“HIFU”), etc. In contrast, electroporation and/or irreversible electroporation is a primarily non-thermal therapy, and has significant potential benefits over thermal modalities. Energy may be applied to perform electroporation and/or irreversible electroporation (“IRE”) as a mode of treating various conditions and/or diseases using an electric field to interrupt and/or to change the nature of biological cellular matter. For instance, the applied electric field may significantly increase the electrical conductivity and permeability of the plasma in the cell membrane. The applied energy causes paths/pores to open within cell walls and/or membranes near the device applying the energy (e.g., near the electrode, probe, etc., thereof). The electric field disrupts homeostasis, and, in the case of IRE, kills the cells, such as through apoptosis and/or necrosis. Monopolar probes may be used to insert a single electrode/an electrode of a first polarity (typically an anode) within the patient, with energy directed to the second, ground electrode typically outside the patient (e.g., a grounding pad positioned on the skin of a patient). However, such configuration may have certain effects, such as muscle stimulation such as which may result in muscle contractions; cardiac interference, etc., which may not be desirable. To mitigate such effects, bipolar probes with two electrodes, typically separated by insulation, yet positioned close enough to create an energy field therebetween with fewer effects than caused by exposure to electrical fields created by monopolar probes. Once positioned within the target site (e.g., a tumor), the monopolar probe is activated, such as by generating an electric field between and/or around the electrodes of the device, for various therapeutic procedures. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a multi-electrode energy-delivering assembly is formed with at least one or more electrodes of a first polarity; and a support structure coupling the one or more electrodes for delivery as an assembly within a patient. In some aspects, the multi-electrode energy delivering assembly has an elongated compact delivery configuration for delivery through a lumen of a delivery device.

In some aspects, the one or more electrodes include two or more electrodes of the first polarity axially spaced from one another and extending in a linear configuration when in the elongated compact delivery configuration. In some aspects, the two or more electrodes are formed from an elongate electrically-conductive member and defined by an insulation member extending over and exposing portions of the elongate electrically-conductive member. In some aspects, the multi-electrode energy-delivering assembly further includes at least one electrode of a second polarity axially spaced from at least one of the two or more electrodes of the first polarity. In some aspects, the at least one electrode of a second polarity is formed from a tubular electrically-conductive member positioned over the insulation member. In some aspects, the multi-electrode energy-delivering assembly further includes at least one differentiating insulation member extending over a portion of the tubular electrically-conductive member to define two or more spaced apart electrodes of the second polarity.

In some aspects, the two or more electrodes are positioned on a support element expandable from the elongated delivery configuration into an expanded deployed configuration when extended out of a delivery device to increase the volume of an electric field to be defined by the electrodes when the support element is in the deployed configuration compared with an electric field which would be defined by the electrodes when the support element is in the delivery configuration.

In some aspects, the one or more electrodes include three or more electrodes movable with respect to one another. In some aspects, at least one of the three or more electrodes are configured to engage and remain engaged with tissue.

In some aspects, the one or more electrodes of a first polarity comprises a fluid containing electrically-conductive particles injectable by the delivery device into a target site within a patient. In some aspects, the multi-electrode energy-delivering assembly further includes an additional electrode of a second polarity delivered to the target site with the fluid.

In some aspects, the multi-electrode energy-delivering assembly is expandable upon deployment from a delivery device.

In accordance with various principles of the present disclosure, a multi-electrode energy-delivering treatment system includes a multi-electrode energy-delivering assembly, a power connector configured to deliver energy to one or more electrodes of the multi-electrode energy-delivering assembly to generate an electric field among the one or more electrodes; and a tubular delivery device having a lumen within which the multi-electrode energy-delivering assembly is deliverable to a target site within a patient. In some aspects, one or more electrodes of the multi-electrode energy-delivering assembly are of a first polarity; and the multi-electrode energy-delivering assembly further includes a support structure coupling the one or more electrodes for delivery as an assembly within a patient. In some aspects, the multi-electrode energy delivering assembly is delivered within the lumen of the delivery device in an elongated compact delivery configuration.

In some aspects, the multi-electrode energy-delivering assembly is expandable upon deployment from the lumen of the delivery device.

In some aspects, the one or more electrodes of the multi-electrode energy-delivering assembly include two or more electrodes extending axially spaced apart from one another when in the lumen of the delivery device.

In accordance with various principles of the present disclosure, a method of applying therapeutic energy to a patient includes delivering, to a target site within a patient, a plurality of electrodes of a first polarity in an elongated delivery configuration within a lumen defined in a delivery device; deploying the plurality of electrodes out of the lumen of the delivery device; and delivering energy to the deployed plurality of electrodes to create an energy field between the plurality of electrodes.

In some aspects, the method further includes spacing apart the electrodes of the plurality of electrodes from one another upon deployment thereof. Additionally or alternatively, the method further includes activating the plurality of electrodes independently of one another.

In some aspects, the plurality of electrodes includes a fluid containing a plurality of electrically-conductive particles, and the method further includes delivering the plurality of electrodes by injecting the fluid into the patient at the target site.

In some aspects, the delivery device includes an electrically-conductive material, and the method further includes delivering energy to the delivery device to generate an electric field between the electrically-conductive material of the delivery device and the electrically-conductive particles in the fluid.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of an example of an embodiment of an energy-delivering treatment system formed in accordance with various aspects of the present disclosure.

FIG. 2 illustrates an elevational view of an example of an embodiment of a multi-electrode energy-delivering assembly formed in accordance with various aspects of the present disclosure.

FIG. 3 illustrates an elevational view of an example of an embodiment of a multi-electrode energy-delivering assembly similar to the embodiment illustrated in FIG. 2, but with fewer electrodes.

FIG. 4 illustrates a perspective view of an example of an embodiment of a multi-electrode energy-delivering treatment system formed in accordance with various aspects of the present disclosure, with enlarged views of examples of embodiments of electrodes thereof.

FIG. 5A, FIG. 5B, and FIG. 5C illustrate elevational views of an example of an embodiment of a multi-electrode energy-delivering assembly with at least one electrode in a fluid formed in accordance with various aspects of the present disclosure.

FIG. 6 illustrates a perspective view of an example of an embodiment of a multi-electrode energy-delivering assembly having a plurality of elongated electrodes formed in accordance with various aspects of the present disclosure.

FIG. 6A illustrates a cross-sectional view along line VIA-VIA of FIG. 6.

FIG. 7 illustrates a perspective view of an example of an embodiment of a multi-electrode energy-delivering assembly similar to that of FIG. 6, with modified electrodes formed in accordance with various aspects of the present disclosure.

FIG. 7A illustrates a cross-sectional view along line VIIA-VIIA of FIG. 7.

FIG. 8A illustrates an elevational view of an example of an embodiment of a multi-electrode energy-delivering treatment system formed in accordance with various aspects of the present disclosure and in a compact delivery configuration.

FIG. 8B illustrates an elevational view of a multi-electrode energy-delivering assembly similar to that of FIG. 8A, but in an expanded configuration such as for deployment into tissue at a target site.

FIG. 8C illustrates an elevational view of a multi-electrode energy-delivering assembly similar to that of FIG. 8A, but in an expanded configuration such as for deployment within a body lumen.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As understood herein, corresponding is intended to convey a relationship between components, parts, elements, etc., configured to interact with or to have another intended relationship with one another. As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. For the sake of convenience, reference may be made to terms such as therapy, treatment, diagnosis, procedure, etc., including various grammatical forms thereof, alternately and without intent to limit, reference to one such term not excluding the others unless explicitly stated. Moreover, it will be appreciated that reference may be made herein to a treatment site, target site, site, etc., interchangeably and without intent to limit. Finally, reference to “at” a location or site is intended to include at and/or about the vicinity of (e.g., along, adjacent, proximate, etc.) such location or site.

As generally used herein, the term “ablation” generally refers to removal of cells either directly or indirectly by supply of energy within an electric field and may include removal by loss of cell function, cell lysis, coagulation, protein denaturation, necrosis, apoptosis, and/or irreversible electroporation. “Ablation” may similarly refer to creation of a lesion by ablation. Additionally, the terms “undesirable tissue,” “target cells,” “diseased tissue,” “diseased cells,” “tumor,” “cell mass” may be used herein to refer to cells removed or to be removed, in whole or in part, by ablation, and are not intended to limit application of any assemblies, systems, devices, or methods described herein. For example, such terms include ablation of both diseased cells and certain surrounding cells, despite no definite indication that such surrounding cells are diseased. Ablation performed by assemblies, systems, devices, or methods described herein may be of cells located around a biological lumen, such as a vascular, ductal, or tract area, for example, to create a margin for a medical professional to resect additional cells by ablation or other method. In accordance with various principles of the present disclosure, devices, assemblies, systems, and methods disclosed herein may be configured for performing ablation via electroporation and/or IRE.

Electrical ablation devices accordance with various principles of the present disclosure and described herein include two or more, and even three or more, electrodes configured to be positioned into or proximal to undesirable tissue in a tissue treatment region (e.g., a target site or a worksite). The tissue treatment region may have evidence of abnormal tissue growth. In general, the electrodes may include an electrically conductive portion and may be configured to be electrically coupled to an energy source. In some aspects, the devices, assemblies, systems, and methods of the present disclosure are configured for use in electroporation and/or irreversible electroporation (“IRE”) treatments/therapies. For instance, devices, assemblies, systems, and methods may be configured in accordance with various principles of the present disclosure for minimally invasive ablation treatment of undesirable tissue through the use of IRE. Minimally invasive ablation treatment may be characterized by the ability to ablate selected tissue in a controlled and focused manner with reduced or no thermally-damaging effects to surrounding healthy tissue.

In accordance with various principles of the present disclosure, an energy-delivering treatment system (such as capable of performing electroporation and/or IRE) has a multi-electrode-energy-delivering assembly with two or more electrodes across which therapeutic energy is transferred, generally delivered and deployed together at/to/within/around a target site/treatment site within a patient's body. In some aspects, multi-electrode-energy-delivering assemblies formed in accordance with various principles of the present disclosure are bipolar or unipolar devices with at least one cathode and at least one anode positioned within the patient. In some aspects, the one or more cathodes and/or the one or more anodes are positioned within the patient in a manner configured to mitigate effects of monopolar devices. In some aspects, the electrodes are arranged to deliver therapeutic energy to a patient across the electrodes of the assembly, thereby limiting energy fields to the region of the electrodes. As such, transmission of energy through the patient's body is more confined than when similar amounts and types of energy are delivered with a monopolar device with a single electrode at the treatment site and utilizing another electrode (typically a return or grounding pad) positioned a distance away from the single electrode (typically outside the patient). Accordingly, in contrast with monopolar devices, the multi-electrode energy-delivering assemblies described herein may be considered to function as a bipolar or unipolar device (or devices) which mitigates some undesirable effects of monopolar probes used with living organisms such as human patients. And in contrast with various bipolar probes, multi-electrode energy-delivering assemblies disclosed herein include two or more electrodes, and even three or more electrodes, coupled together for delivery as an assembly/as a unit. Additionally or alternatively, various of the multi-electrode energy-delivering assemblies described herein have a compact delivery configuration facilitating deliver of the multi-electrode energy-delivering assembly as an assembly/as a unit, through a lumen or working channel of a delivery device, and/or percutaneously, and/or individually through luminal spaces in the patient's body. For instance, the electrodes of the multi-electrode energy-delivering assembly may be along a common member of the multi-electrode energy-delivering assembly. In some aspects, the multi-electrode energy-delivering assembly has a plurality of electrodes formed from an elongate electrically conductive element with portions/sections/regions thereof separated to define spaced apart electrodes therealong. In some aspects, the multi-electrode energy-delivering assembly includes a separate element, such as a support element, on which electrodes are mounted. The two or more electrodes may be generally axially spaced apart from one another, such as along an elongated common member. For instance, in some aspects, an electrode of a given polarity may extend along a support element such as an electrode of a different polarity, and/or a support element which may otherwise affect an energy field created with respect to the multi-electrode energy-delivering assembly. In some aspects, the two or more electrodes are delivered together as an assembly/as a unit generally coupled together, yet also movable with respect to one another. In some aspects, the two or more electrodes are delivered in a generally elongated configuration facilitating delivery through a lumen or working channel of a delivery device.

To effectively treat a target site with electroporation and/or IRE, an effective electric field must be created between the electrodes of a multi-electrode-energy delivering assembly formed in accordance with various principles of the present disclosure. More particularly, in the case of a bipolar probe, a first electrode of a first polarity must be electrically insulated from a second electrode of a second polarity. In accordance with various principles of the present disclosure, either or both electrodes of a multi-electrode-energy delivering assembly formed in accordance with various principles of the present disclosure are formed of an electrically-conductive material (e.g., medical grade stainless steel, platinum, gold, nitinol, a cobalt-chromium alloy, a nickel-cobalt alloy such as MP35N, copper, or other alloys, or materials plated with electrically-conductive materials, etc.). In some aspects, an insulation member is positioned between the electrodes, such as to electrically isolate the electrodes (typically with one electrode serving as an anode and another electrode serving as a cathode). The insulation member may be formed of any appropriate insulative material (e.g., a polymer extrusion, a polymer heat shrink, ceramic, a composite shaft, such as formed of fluoropolymer, polyimide, polyethylene terephthalate (PET), nylon, and/or any other suitable material of adequate dielectric strength and biocompatibility, and any combinations or composites thereof, etc.) capable of isolating electrodes of opposite polarities.

The various multi-electrode energy-delivering assemblies described herein may be deliverable through an elongate tubular member (e.g., a delivery sheath, catheter, working channel of an endoscope, etc.) inserted into a patient (such as through a natural anatomical passage or orifice and into a body lumen within a patient), or transcutaneously or percutaneously. The energy-delivering member of the energy-delivering assembly may be coupled to an energy source to energize the electrode portion thereof to apply an electric current to biological tissue. The energy source may be operative to generate an electric field between the electrode portion and another electrode portion, such as an electrode portion coupled to the energy source and having an opposite polarity, e.g., a return or ground. Once an energy-delivering assembly formed in accordance with various principles of the present disclosure is positioned at or near undesirable tissue, an energizing potential may be applied to the electrode portions thereof, such as to create an electric field to which the tissue at the target site is exposed. The energizing potential (and the resulting electric field) may be characterized by various parameters, such as, for example, frequency, amplitude, pulse width (duration of a pulse or pulse length). Suitable energy sources include electrical waveform generators, such as waveform generators capable of creating IRE, high frequency IRE, pulsed, nanopulse, and/or ablative waveforms. The energy source generates an electric field with desired characteristics for the treatment to be performed at the target site. For instance, the electric field may be generated to have suitable characteristic waveform output in terms of voltage, impedance, frequency, amplitude, pulse width, delays between pulses, number of pulses per burst, number of bursts, and polarity. Optionally, the pulse sequences are triggered in accordance with the patient's cardiac cycle (e.g., so as not to interfere with the patient's cardiac cycle). The electric current flows between the electrodes and through the tissue proportionally to the potential (e.g., voltage) applied to the electrodes. The supplied electric current provided by the energy source may deliver a pulse sequence to the target site. For example, an energy source may supply various waveforms in one or more pulse sequences tailored to the desired application.

Energy-delivering assemblies, devices, systems, and methods described herein may be utilized for electroporation, irreversible electroporation (IRE), reversible electroporation, and/or electropermeabilization techniques to apply external electric fields (electric potentials) to cell membranes to significantly increase permeability of the plasma membrane of the cell, such as to improve uptake of therapeutic materials by the cell. Optionally, the energy applied to the cell may change the characteristics of the cell membranes (e.g., porosity), such as irreversibly, resulting in cell death (e.g., by apoptosis and/or necrosis). Such techniques may advantageously be used to treat/apply therapy without raising the temperature of the surrounding tissue to a level at which permanent damage may occur to the surrounding tissue, support structure, and/or regional vasculature. Application of IRE pulses to cells may thus be an effective way for ablating large volumes of undesirable tissue with no or minimal detrimental thermal effects to the surrounding healthy tissue.

Various embodiments of electrode devices, assemblies, systems, and various associated methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered.

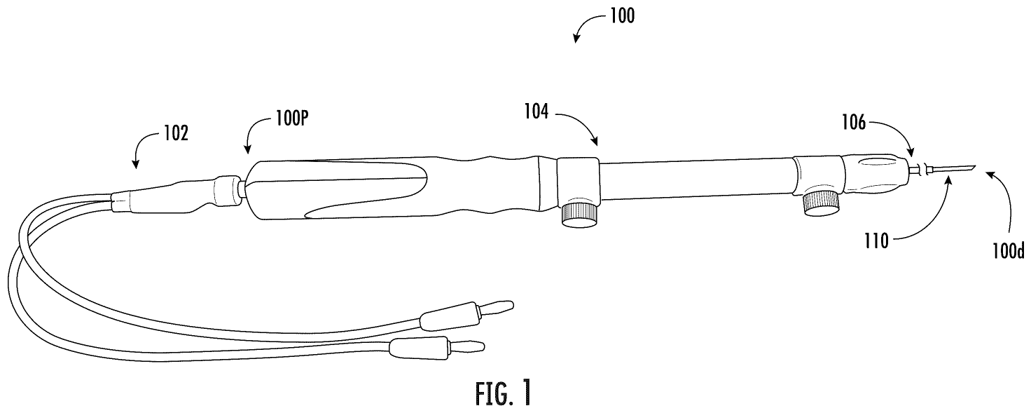

Turning now to the drawings, an example of an embodiment of an energy-delivering treatment system 100 is illustrated in FIG. 1. The energy-delivering treatment system 100 includes a multi-electrode energy-delivering assembly 110 (illustrated schematically in FIG. 1) extending along the distal end 100d of the energy-delivering treatment system 100. The multi-electrode energy-delivering assembly 110 is configured to deliver energy to a target site on/in a patient. As such, an energy source is coupled to the proximal end 100p of energy-delivering treatment system 100. The multi-electrode energy-delivering assembly 100 may include a power connector 102, such as wiring configured to be coupled to an energy source such as known those of ordinary skill in the art and selectable by known means based on the type of energy to be applied by the multi-electrode energy-delivering assembly 100. For instance, the energy source may be selected, in a manner known to those of ordinary skill in the art, to apply energy of a nature and in a manner to energize the energy-delivering assembly 110 to be used for electroporation and/or IRE. In some aspects, the energy-delivering equipment has the ability to multiplex the output signal to individual electrode anode and cathode pairs or multiple anodes to a single cathode selectively, and/or the ability to time the energy delivery to the cardiac cycle. The present disclosure need not be limited by the details of the energy source.

In some aspects, the energy-delivering assembly 110 is delivered to a target site within a patient through a delivery device, such as a tubular element (e.g., catheter, sheath, endoscope, etc.) having a lumen or working channel therethrough sized to allow passage of the energy-delivering assembly 110 therein/therethrough. Such delivery device may be selected from a variety of delivery devices known to those of ordinary skill in the art, the present disclosure not being limited in this regard. Optionally, a protective sheath 106 is provided over the multi-electrode-energy delivering assembly 110 to protect the delivery device from a sharp distal tip or other features of the multi-electrode-energy delivering assembly 110. Such protective sheath 106 may be in a form generally known those of ordinary skill in the art such that illustration thereof is not necessary for one of ordinary skill in the art to understand the nature of such member.

The energy-delivering treatment system 100 optionally further includes a handle 104 operatively coupled with the multi-electrode energy-delivering assembly 110, such as to control elements of the multi-electrode energy-delivering assembly 110. For instance, the handle 104 may be configured to control the relative positions of the multi-electrode energy-delivering assembly 110 with respect to a delivery device and/or protective sheath, and/or to control and/or to adjust the position of one or more elements of the energy-delivering assembly 110.

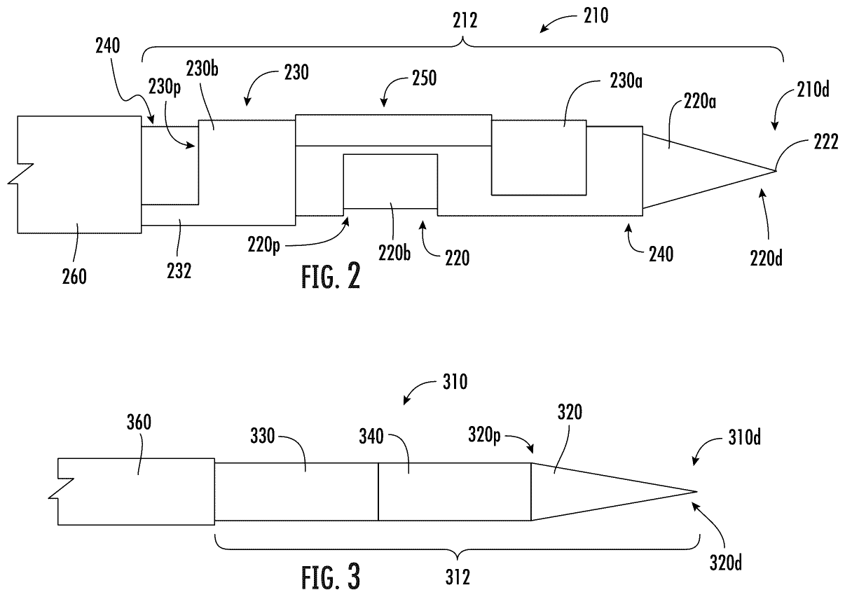

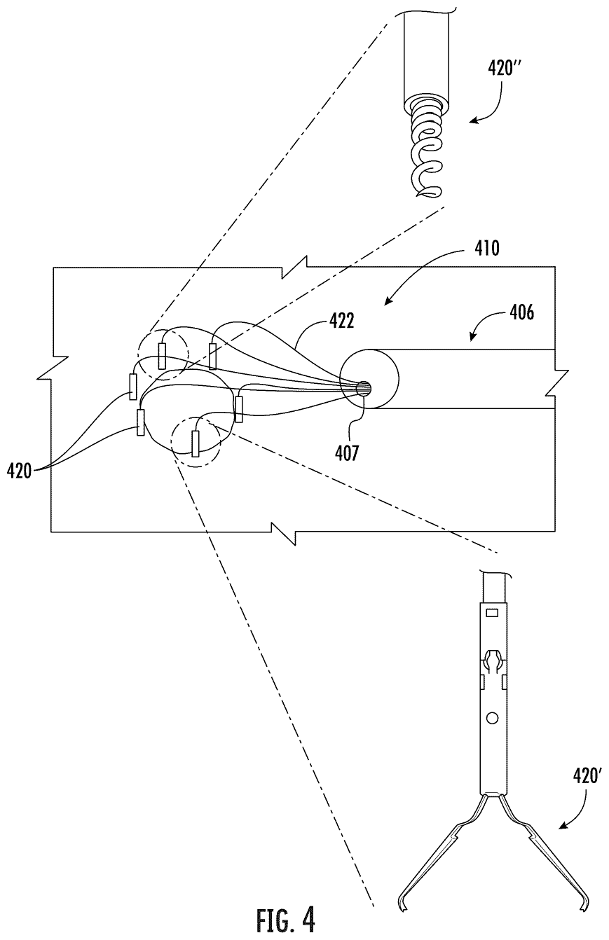

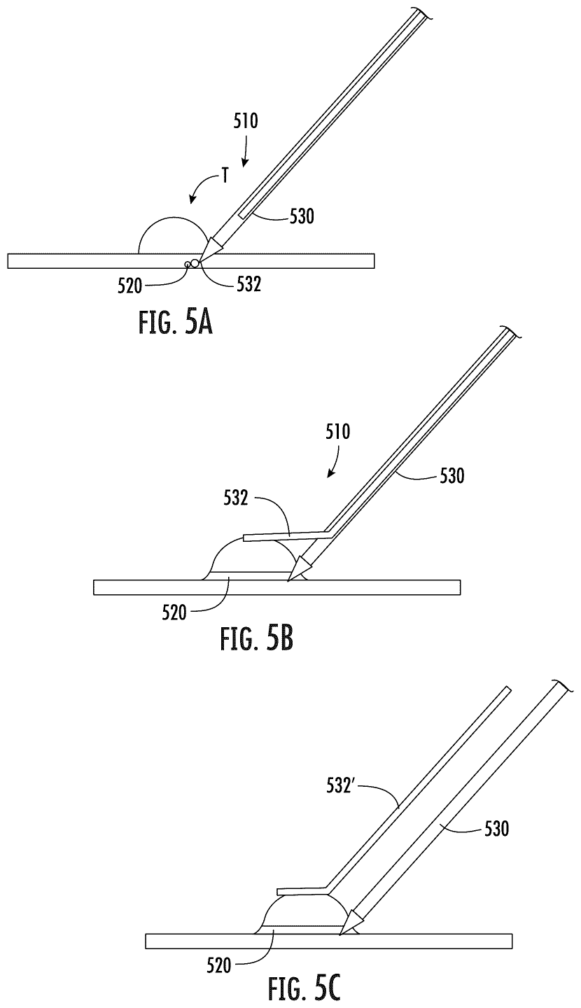

In accordance with various principles of the present disclosure, as discussed briefly above, a multi-electrode-energy delivering assembly formed in accordance with various principles of the present disclosure has two or more electrodes to be delivered to a target site on/within a patient. The electrodes are arranged and/or configured such that application of energy thereto creates a field of therapeutic energy which is to be applied to the target site in accordance with various principles of the present disclosure. Various formations of multiple electrodes of multi-electrode-energy delivering assemblies formed in accordance with various principles of the present disclosure are illustrated in FIG. 2, FIG. 3, FIG. 4, FIGS. 5A-5C, FIG. 6, FIG. 6A, FIG. 7, FIG. 7C, and FIGS. 8A-8C.

The example of an embodiment of a multi-electrode-energy delivering assembly 210 illustrated in FIG. 2, the multi-electrode-energy delivering assembly 210 includes a first energy-delivering member 220 formed of an electrically-conductive material such as medical grade stainless steel, platinum, gold, nitinol, copper, a cobalt-chromium alloy, a nickel-cobalt alloy such as MP35N, or other alloys, or materials plated with electrically-conductive materials, etc. In accordance with various principles of the present disclosure, at least the first energy-delivering member 220 of the multi-electrode-energy delivering assembly 210 illustrated in FIG. 2 is an elongate (flexible or rigid) element capable of being navigated through a patient's body, such as through natural orifices and/or through a lumen of a tubular elongate members inserted into the patient's body. More particularly, the first energy-delivering member 220 may be elongate and sufficiently flexible to be able to be inserted transluminally into the body (e.g., in contrast with being percutaneously inserted) and navigated through potentially tortuous pathways within the body, or at least being capable of bending or turning with/within natural, nonlinear anatomical structures. Additionally or alternatively, the first energy-delivering member 220 may be sufficiently resilient so as not to break as it is being navigated. It will be appreciated that those of ordinary skill in the art may determine appropriate length, flexibility, resiliency, deliverability (e.g., atraumatic shape, torquability, pushability, trackability, deployability, retractability, etc.) and/or other properties/characteristics of a first energy-delivering member 220 used in accordance with various principles of the present disclosure based on the material, size, shape, configuration, and/or dimensions of the first energy-delivering member 220, the present disclosure not necessarily being limited to specific parameters. In some aspects, the first energy-delivering member 220 has a sharp distal tip 222 at a distal end 220d thereof for piercing/puncturing tissue, such as to achieve better access to a target site within a patient. The elongate element may be a trocar (e.g., a solid element), or a tubular element such as a needle configured to irrigate/aspirate materials with respect to the target site.

In accordance with various principles of the present disclosure, the multi-electrode-energy delivering assembly 210 is a bipolar assembly. As understood herein and as by those of ordinary skill in the art, a bipolar assembly has an anode as well as a cathode/ground electrode positioned and delivered together at/to within/around a target site/treatment site within a patient's body. Such configuration may be readily understood by those of ordinary skill in the art to be in contrast with a monopolar probe which delivers only electrodes of one polarity to a target site, with the opposite polarity electrode delivered at a different anatomical location with respect to the target site, such as outside the body (in contrast with the monopolar probe delivered to a target site within the body). In the example of an embodiment of a multi-electrode-energy delivering assembly 210 illustrated in FIG. 2, the electrodes thereof are provided along a distal end 210d of the multi-electrode-energy delivering assembly 210 for delivery to a target site with respect to a patient to deliver therapeutic/treatment energy to a patient.

In accordance with various principles of the present disclosure, the first energy-delivering member 220 of the example of an embodiment of a multi-electrode-energy delivering assembly 210 illustrated in FIG. 2 may be considered to form a first electrode of a first polarity (i.e., an anode or a cathode of the multi-electrode-energy delivering assembly 210) of the bipolar multi-electrode-energy delivering assembly 210. As may be appreciated, the first energy-delivering member 220 is electrically coupled with an appropriate energy source (such as generally described above), such as in a manner known those of ordinary skill in the art. For instance, the energy source may be coupled with the first energy-delivering member 220 via a power connector 102, such as described above, either directly or via leads extending therebetween, such as outside the patient. For the sake of convenience, and without intent to limit, the first energy-delivering member 220 may be described herein as forming a primary electrode of the example of an embodiment of a multi-electrode energy-delivering assembly 210 illustrated in FIG. 2.

The multi-electrode-energy delivering assembly 210 illustrated in FIG. 2 is a multi-electrode probe with multiple segments of alternating polarity along the length thereof. In the example of an embodiment of a multi-electrode-energy delivering assembly 210 illustrated in FIG. 2 the above-described first energy-delivering member 220 has a first polarity, and a second energy-delivering member 230 is provided with a polarity opposite the polarity of the first energy-delivering member 220. The example of an embodiment of a second energy-delivering member 230 illustrated in FIG. 2 is in the form of an outer electrically-conductive element positioned over (e.g., on top of and/or circumferentially around/surrounding) an underlying electrically conductive element (e.g., the first energy-delivering member 220). The example of an embodiment of a second energy-delivering member 230 illustrated in FIG. 2 is in the form of an electrically-conductive tubular element extending over/on top of at least a portion of the underlying first energy-delivering member 220. The second energy-delivering member 230 may be formed from a hypotube; a laser-cut hypotube; and/or a sheet of material rolled and/or formed or otherwise positioned over and/or conformed to the outer dimension/surface of the first energy-delivering member 220. Alternatively or additionally, the second energy-delivering member 230 may be formed by sputtering, photodeposition, exposing underlying conductor(s)/electrode(s) by removing encapsulating material (such as a composite shaft design), and/or another material deposition technique by which an electrically-conductive material may be provided over another element.

In accordance with various principles of the present disclosure, an insulation layer or member 240 is provided between the first energy-delivering member 220 and the second energy-delivering member 230 to electrically isolate/insulate the electrically-conductive materials thereof from each other. The insulation member 240 may be formed of a layer and/or coating of insulative material (e.g., such as an insulative material as described above) which may be selected based on the material of the first energy-delivering member 220 and/or the second energy-delivering member 230. In some aspects, the insulation member 240 may be cut or etched away to reveal/expose the underlying material of the first energy-delivering member 220 to serve as an electrode of the multi-electrode-energy delivering assembly 210. For instance, as illustrated in FIG. 2, the insulation member 240 may leave exposed two regions of the underlying first energy-delivering member 220 spaced apart from each other to differentiate a first electrode 220a and a second electrode 220b of the same polarity (as the first energy-delivering member 220). The insulation member 240 otherwise generally extends proximally from the proximal end 220p of the proximal-most electrode (in the illustrated example of an embodiment, the second electrode 220b) formed of the first energy-delivering member 220, such as up to the connection of the first energy-delivering member 220 to an energy source. As such, the insulation member 240 insulates the patient from energy delivered from an energy source coupled to a proximal end of the first energy-delivering member 220. Additionally or alternatively, the insulation member 240 may delimit an energy-delivery region 212 of the energy-delivering member 220 and the multi-electrode-energy delivering assembly 210 adjacent the distal end 220d of the first energy-delivering member 220 and the distal end 210d of the multi-electrode-energy delivering assembly 210 (e.g., with the use of additional insulative materials).

Optionally, if the second energy-delivering member 230 is formed of a single, generally continuous element extending over the first energy-delivering member 2220 (such as a hypotube or cut hypotube), a differentiating insulation member 250 may be provided over one or more portions/regions/sections (such terms being used interchangeably herein without intent to limit unless otherwise specified) to separate and define portions of the second energy-delivering member 230 as separate electrodes 230a, 230b (of the same polarity, opposite that of the first energy-delivering member 220). The differentiating insulation member 250 may be formed of an insulative material such as ceramic, polymer, etc., such as may be selected based on the material of the underlying second energy-delivering member 230.

As may be appreciated, the second energy-delivering member 230 is a different polarity than that of the first energy-delivering member 220, and thus is electrically-connected at its proximal end as well, such as in a manner known those of ordinary skill in the art. In the example of an embodiment illustrated in FIG. 2, an electrical connector, an extension of the second energy-delivering member 230, or a lead 232 extends proximally from the second energy-delivering member 230 (e.g., from the proximal electrode 230b thereof) to electrically couple the second energy-delivering member 230 with a grounding electrode or an energy source (e.g., via a connector 102 such as illustrated in FIG. 1, such as outside the patient). However, other configurations are within the scope and spirit of the present disclosure, such as other forms of proximal extensions of the material forming the second energy-delivering member 230. It will be appreciated that an insulation jacket 260 may be provided over a proximal extent of the electrical lead 232 extending proximally from the second energy-delivering member 230 to electrically-insulate the electrical lead 232. Such insulation jacket 260 may be advantageous if at least a portion of the second energy-delivering member 230 is extended proximally to an energy source/ground. Additionally or alternatively, the insulation member 260 may delimit an energy-delivery region 212 of the energy-delivering members 230 and 220 and the multi-electrode-energy delivering assembly 210. Additionally or alternatively, an insulation member 250 may be applied over the electrical connector or lead 232 to delimit an energy-delivery region 212 of the energy-delivering members 230 and 220 and the multi-electrode-energy delivering assembly 210. It should be appreciated that instead of energy being supplied to the first energy-delivering member 220 with the second energy-delivering member 230 being grounded, the reverse arrangement is also acceptable. Additionally or alternatively, the first energy-delivering member 220 with the second energy-delivering member 230 may be electrically coupled with an electrode/ground pad on the outside of the patient or another device(s) within the patient.

In accordance with various principles of the present disclosure, the example of an embodiment of a multi-electrode energy-delivering assembly 210 illustrated in FIG. 2 is configured to allow for multiple segments of alternating polarity along the length thereof. With one or more cut out areas of the insulation member 240 overlying the first energy-delivering member 220, with one or more cut out areas of the conductive energy-delivering member 230 overlying the insulation member 240, and with one or more differentiating insulation members 250, a multi-electrode configuration may be achieved relatively simply in accordance with various principles of the present disclosure to form a linear multi-electrode probe capable of delivering energy for performing a therapeutic procedure such as electroporation and/or irreversible electroporation. It will be appreciated that a linear multi-electrode-energy delivering assembly 210 such as illustrated in FIG. 2 provides a smooth and improved overall ablation shape relative to prior bipolar probes (which typically have separate and sometimes independently movable probes). Alternatively or additionally, a linear multi-electrode energy-delivering assembly 210 such as illustrated in FIG. 2 may reduce the power need for achieving the desired therapeutic effect upon delivering energy thereto. For instance, less power may be needed for an equivalent ablation size with a configuration as illustrated in FIG. 2 in comparison with a monopolar configuration with a ground pad external to the patient. Additionally or alternatively, a multi-electrode energy-delivering assembly 210 such as illustrated in FIG. 2 may allow the shaping and sizing of an ablation zone based on the positioning and/or sizing of the electrode(s) in relation to one another.

It will be appreciated that the first energy-delivering member 220, and/or the second energy-delivering member 230, and/or the insulation member 240 therebetween, and/or the differentiating insulation member 250 may have alternative configurations than those illustrated in FIG. 2 while achieving the same or similar benefits in accordance with various principles of the present disclosure. For instance, in the example of an embodiment of a multi-electrode-energy delivering assembly 310 illustrated in FIG. 3, only a distal region 320d of the first energy-delivering member 320 is exposed to form a first electrode adjacent the distal end 310d of the multi-electrode-energy delivering assembly 310. Moreover, only a single region of additional electrically-conductive material may be exposed over the insulation member 340 (and thus electrically insulated from the first energy-delivering member 320) to form a single second energy-delivering member 330 of a polarity different from that of the first energy-delivering member 320. Alternatively, more than the two electrodes of each polarity, as illustrated in the example of an embodiment of a multi-electrode-energy delivering assembly 210 of FIG. 2, may be formed/provided in a multi-electrode-energy delivering assembly formed in accordance with various principles of the present disclosure. Formation of more than two electrodes of each polarity may result in smoother electric fields formed around the individual electrodes, which may result in a smoother, more even profile across the length of the multi-electrode-energy delivering assembly. Such configurations are readily understood by those of ordinary skill in the art and thus illustration thereof is not necessary, and has not been provided for the sake of brevity.

In accordance with various principles of the present disclosure, a multi-electrode-energy delivering assembly may be advantageously formed in other than linear configurations, with the various electrodes thereof movable with respect to one another (in contrast with the electrodes 220, 230 of the above-described multi-electrode-energy delivering assembly 210 which are generally fixed with respect to each other). For instance, the example of an embodiment of a multi-electrode-energy delivering assembly 410 illustrated in FIG. 4 has a plurality of individual, separately and independently positionable electrically-conductive electrodes 420 which are positioned strategically with respect to a target site to apply therapeutic energy thereto. The electrodes 420 may be any of a variety of configurations capable of engaging tissue, such as clips (such as the illustrated tissue clip 420′), tissue spirals (such as the illustrated spiral/helical tissue-engaging or anchoring element 420″), fiducials, or other tissue-engaging elements known to those of ordinary skill in the art. The electrodes 420 or at least a portion thereof may be formed of an electrically-conductive material (such as any of those described above).

In some aspects, the example of an embodiment of a multi-electrode-energy delivering assembly 410 illustrated in FIG. 4 may be delivered within a lumen 407 of a delivery device 406 (such as an endoscope). In some aspects the electrodes 420 are delivered together, coupled together as an assembly or a unit, with the electrodes 420 coupled to define, together, an energy field such as ablation zone. More particularly, in some aspects, the electrodes 420 are delivered and deployed together at/to/within/around a target site/treatment site within a patient's body. The electrodes 420 are deployed out of the delivery device 406 with the leads 422 extending proximally through a lumen/working channel 407 of the delivery device 406. In some aspects, the delivery device 406 may be considered a part of the energy-delivering treatment system of the present disclosure, such as the energy-delivering treatment system 100 illustrated in FIG. 1.

Each of the electrodes 420 may be individually and separately connected to an energy source (e.g., via a power connector 102, such as illustrated in FIG. 1), such as via one or more leads 422 (which may be coupled to the energy source outside the patient). The leads 422 of the electrodes 420 may be independent of (e.g., electrically insulated from) one another. Optionally, one or more leads 422 may be connected/electrically coupled. At least one of the leads 422 is electrically coupled with an energy source to deliver therapeutic energy to the one or more electrodes 420. The electrodes 420 may be deployed using a device similar to a clip, a fine needle aspiration (FNA) needle, or an endoscopic suturing device. In some aspects, each of the electrodes 420 may be activated independently. Alternatively or additionally, some or all of the electrodes 420 may be activated simultaneously. Alternatively or additionally, the electrodes 420 may form two groups, one group connected to the anode and the other to the cathode.

It will be appreciated that a multi-electrode-energy delivering assembly 410 formed in accordance with various principles of the present disclosure and configured as illustrated in FIG. 4 allows for a custom ablation profile. Alternatively or additionally, the example of an embodiment of a multi-electrode-energy delivering assembly 410 illustrated in FIG. 4 may be used on the surface of anatomical structures (e.g., organs) or deeper within tissue (e.g., within a tumor). Moreover, a multi-electrode-energy delivering assembly 410 such as illustrated in FIG. 4 may facilitate delivery of therapeutic energy over a volume significantly larger than the delivery device by which a single-electrode-energy delivering assembly is delivered and deployed. For instance, the various electrodes 420 of the multi-electrode-energy delivering assembly 410 may be delivered individually or in groups through a working channel of a delivery device 406 such as described above, thereby generally not requiring devices larger than currently available delivery devices. Once deployed, the various (two or more, and typically more than two, such as three or more) electrodes 420 may be spaced apart from one another yet still positioned together to define an energy field among the electrodes 420. In some aspects, individual electrodes are independently positionable, yet generally positioned regionally together at a treatment site within the patient. Such energy field may be able to produce larger and/or more customizable energy fields than an energy field created by a device with fixed electrodes, such as which does not expand beyond such size. Additionally or alternatively, the multi-electrode-energy delivering assembly 410 may facilitate treatment around a tumor mass or within a tumor mass, such as based on need.

Another approach to a multi-electrode-energy delivering assembly 510 allowing a degree of adjustability of the electrodes thereof in accordance with various principles of the present disclosure is illustrated in FIG. 5A, FIG. 5B, and FIG. 5C. Instead of a solid structural electrode, the multi-electrode-energy delivering assembly 510 of FIG. 5A, FIG. 5B, and FIG. 5C utilizes an electrically-conductive (and preferably biocompatible) fluid (saline, hydrogel, contrast fluids, and/or other therapeutic fluids) as one electrode (e.g., a virtual electrode activated by an electrical member) of the assembly. The other electrode may be a more traditional structurally solid (in contrast with fluid) electrically-conductive structure. The electrical conductivity of the electrically-conductive fluid 520 may be inherent to the fluid (i.e., the fluid itself is a conductive fluid) and/or achieved by dispersing, distributing, or otherwise providing electrically-conductive particles (e.g., nanoparticles such as gold, platinum, etc., and/or salts to increase fluid conductivity) in the fluid, such as by doping the fluid with electrically-conductive particles.

An example of an embodiment of a multi-electrode-energy delivering assembly 510, such as illustrated in FIG. 5A, FIG. 5B, and FIG. 5C, includes an electrically-conductive fluid 520 delivered by an injection device 530. In accordance with various principles of the present disclosure, and as illustrated in FIG. 5A, the electrically-conductive fluid 520 of the multi-electrode-energy delivering assembly 510 is injected into an anatomical structure (e.g., tissue) at the target site T to which energy is to be applied by an injection device 530 of the multi-electrode-energy delivering assembly 510. The electrically-conductive fluid 520 is schematically illustrated in FIG. 5B as generally delimited, confined, restricted, pooled, bounded, or otherwise localized with respect to a target site T. In some aspects, the electrically-conductive fluid 520 may be a lifting agent which elevates the target site T to facilitate access thereto. In some aspects, the electrically-conductive fluid 520 is re-absorbable and need not be removed by the medical professional or by other active means or steps. Optionally, the electrically-conductive fluid is a viscous fluid or gel with a viscosity selected so that the fluid dwells at the target site T without dissipating through the cellular structure thereof (e.g., the tissue).

The injection device 530 has a lumen defined therethrough in which the electrically-conductive fluid 520 is transported to the target site T and/or from which the electrically-conductive fluid 520 is delivered to the target site T. The injection device 530 may be a needle, hot knife, or other appropriate medical instrument, tool, device, etc. In some aspects, the injection device 530 is formed of an electrically-conductive material (e.g., any of the above described electrically-conductive materials) and forms a second electrode of opposite polarity than the polarity of the first electrode formed by the electrically-conductive fluid 520. The injection device 530 may supply energy to the target site T or may be a return electrode which is grounded, such as depending on materials, structure, nature, and/or other features of the electrically-conductive fluid 520, the injection device 530, and/or the target site T. Optionally, a secondary electrode 532 extends from the injection device 530 and is manipulable with respect to the elongated body of the injection device 530 (e.g., through which the delivery lumen is formed, although the secondary electrode 532 may optionally include a lumen therethrough), such as illustrated in FIG. 5B. Alternatively or additionally, as illustrated in FIG. 5C, a separate and/or additional electrode 532′ may be delivered with the injection device 530 and usable as an additional or alternative second electrode of different polarity than the polarity of the electrically-conductive fluid 520. Optionally, an external grounding pad may be used as a secondary electrode, and/or an additional electrode, and/or ground.

It will be appreciated that the example of an embodiment of a multi-electrode-energy delivering assembly 510 may facilitate delivery of therapeutic energy over a surface area significantly larger than the delivery device by which the multi-electrode-energy delivering assembly 510 is delivered and deployed. For instance, injection of additional electrically-conductive fluid 520 to create a larger energy field (e.g., ablation field) generally does not require an increase in the size of the injection device 530 or any delivery device 406 such as described above with reference to FIG. 4. It may be appreciated that the energy field created by the electrodes of the multi-electrode-energy delivering assembly 510 is larger than an energy field created by a device sized to fit within a lumen of a delivery device and which does not expand beyond such size.

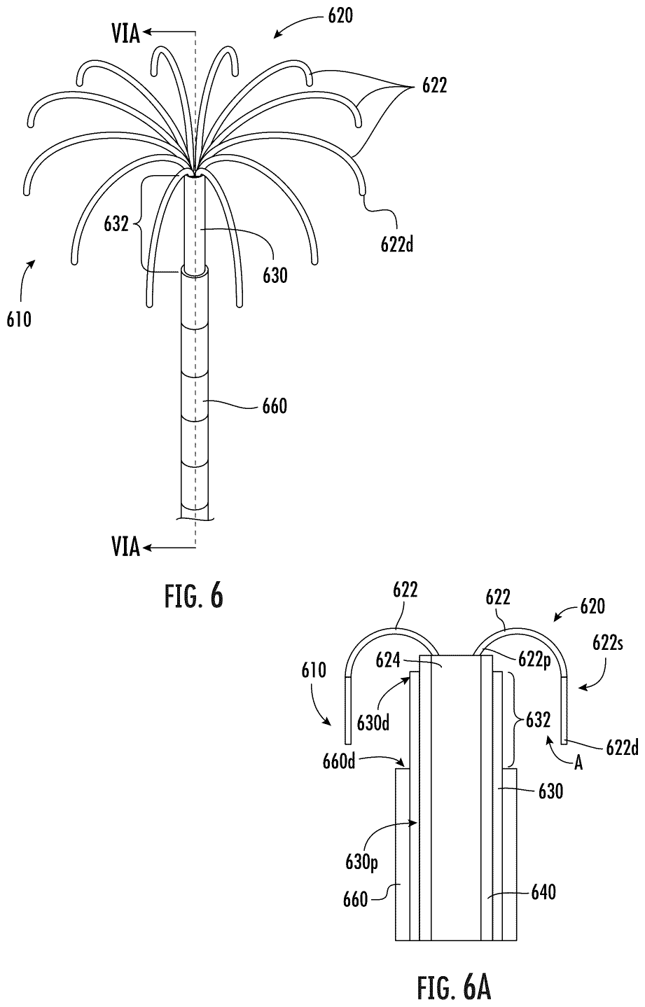

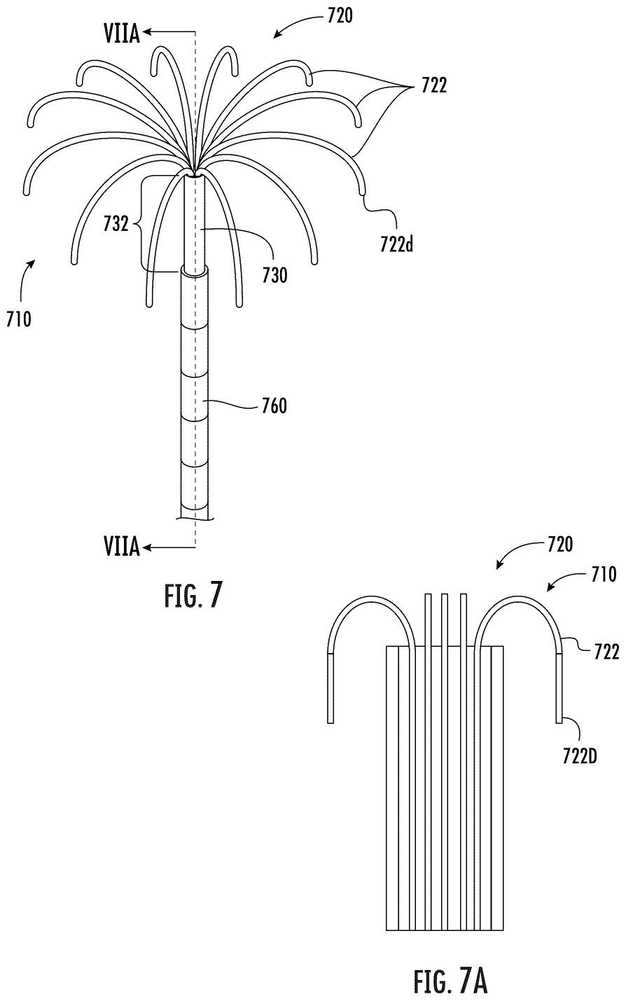

Additional example of embodiments of multi-electrode-energy delivering assemblies with separately formed and/or separately controlled electrodes are illustrated in FIG. 6, FIG. 6A, FIG. 7, and FIG. 7A, illustrating the use of a multi-electrode needle with a plurality of elongated electrodes extending from a shaft. The general configuration of a multi-electrode needle usable in accordance with various principles of the present disclosure may be that of a Le Veen™ Needle Electrode sold by Boston Scientific Corporation, and/or a configuration as described in U.S. Patent Application Publication 2019/0223943, filed Jan. 23, 2019, and titled Enhanced Needle Array And Therapies For Tumor Ablation; and U.S. Patent Application Publication 2020/0129230, filed Oct. 22, 2019, and titled Movable Electrodes For Controlled Irreversible Electroporation Ablative Volumes, which applications are hereby incorporated by reference herein in their entireties and for all purposes.

In the example of an embodiment of a multi-electrode energy-delivering assembly 610 illustrated in FIG. 6, an electrode of a first polarity is formed as a multi-electrode needle 620 having a plurality of electrodes 622. The electrodes 622 may be generally elongated and/or flexible. Optionally, the electrodes 622 may form an umbrella shape with respect to the shaft 624 from which they extend. The electrodes 622 may move from a generally longitudinally extended compact configuration for delivery through a working channel of a delivery device 106 (such as described above with reference to FIG. 4), to a radially-outwardly expanded deployed configuration, such as a straight, angled, or curved (e.g., umbrella) shape/configuration. The deployed configuration may be a pre-set configuration. Optionally, the electrodes 622 are formed from a shape memory, preferably biocompatible, material (e.g., Nitinol, spring tempered steel, etc.) which holds a pre-set configuration into which the multi-electrode energy-delivering assembly 610 shifts upon being deployed. The shape memory material optionally is electrically conductive, or carries an electrically conductive material so that the electrode 622 may create an energy field. Alternatively or additionally, the electrodes 622 may be configured (e.g., free distal terminal ends 622d and/or sides of the electrodes 622 may be configured) to engage and remain engaged with tissue. For instance, sharpened ends or extensions may be provided along the electrodes 622. For instance, in the illustrated example of an embodiment, one or more of the electrodes 622 may end in a sharp distal tip 622d. The electrodes 622 may be referenced herein as electrode tines 622, such as in view of their elongated configuration and/or ability to engage and remain engaged with tissue. In the illustrated example of an embodiment, the electrode tines 622 may be connected on the proximal end to a generally elongated shaft 624. Optionally, the elongated shaft 624 is formed of an electrically-conductive material (such as any of the above-described electrically-conductive materials) through which energy may be conveyed to the electrode tines 622. The elongated shaft 624 may extend proximally from the electrode tines 622 to electrically couple the electrode tines 622 with an energy source (e.g., via a connector 102 such as illustrated in FIG. 1, such as outside the patient). As may be appreciated with reference to the cross-sectional view of FIG. 6A, along line VIA-VIA of FIG. 6, one or more of the electrode tines 622 may have a proximal base end 622p (opposite the distal terminal end 622d thereof) along which the electrode tines 622 extend from the elongated shaft 624.

To form a bipolar multi-electrode energy-delivering assembly 610 in accordance with various principles of the present disclosure, an electrode 630 with a polarity opposite the polarity of the electrode tines 622 is provided in the general vicinity of the electrode tines 622. More particularly, in the example of an embodiment of a multi-electrode energy-delivering assembly 610 illustrated in FIG. 6 and FIG. 6A, a second electrode 630 may be provided over the elongated shaft 624 from which the electrode tines 622 (forming the first electrode of the multi-electrode energy-delivering assembly 610) extend. For instance, the second electrode 630 may be in the form of a tubular electrode extended over (e.g., layered on top of and/or circumferentially around/surrounding the elongated shaft 624) the elongated shaft 624. In some aspects, the elongated shaft 624 from which the electrode tines 622 extend may be proximally retractable with respect to (e.g., into) and/or distally advanceable with respect to (e.g., out of) the tubular electrode 630. For instance, the elongated shaft 624 may be retracted within the tubular electrode 630 during delivery of the multi-electrode energy-delivering assembly 610 to a target site, with the electrode tines 622 in a generally compact configuration (e.g., longitudinally extended distally away from the elongated shaft 624 in generally elongated compact configurations).

In some aspects, the second, tubular electrode 630 is formed of an electrically-conductive material similar to the generally tubular second energy-delivering member 230 illustrated in FIG. 2. As such, reference is made to above descriptions of the second energy-delivering member 230 as generally applicable to the second, tubular electrode 630 illustrated in FIG. 6 and FIG. 6A for the sake of brevity and without intent to limit. Moreover, as may be appreciated with reference to FIG. 6A, in accordance with various principles of the present disclosure, an insulation layer or member 640 is provided between the elongated shaft 624 from which the electrode tines 622 extend (and which may delivery energy to the electrode tines 622 and thus be of a first polarity), and the tubular electrode 630 (which is of a second polarity different from, and typically opposite that of the first polarity) to electrically isolate/insulate the electrode tines 622 and the tubular electrode 630 from one another. Such insulation layer or member 640 may be formed of an insulative material and have a shape/configuration similar to that of the insulation member 240 described above with reference to the multi-electrode energy-delivering assembly 210. As such, reference is made to above descriptions of the second energy-delivering member 230 as generally applicable to the insulation member 640 of FIG. 6 and FIG. 6A for the sake of brevity and without intent to limit. It will be appreciated that the insulation member 640 may be fixed with respect to (e.g., attached, bonded, etc.) to one or both of the elongated shaft 624 and/or the insulation member 640 (such as depending on relative movability therebetween).

As may be appreciated, with the electrode tines 622 and the tubular electrode 630 having different polarities, an ablation zone A may be created/generated in the area between the electrode tines 622 and the tubular electrode 630. To maximize the size and to control the shape of the ablation zone A, the electrode tines 622 may have elongated straight sections (or additional curvature of) 622s extending proximally from the distal ends 622d thereof a distance selected to generate an ablation zone A of the desired size/volume and control the shape such as determined, in part, by the electrical field formed by the electrode tines 622 (e.g., such as may be determined based on various characteristics of the target site to be treated by the multi-electrode energy-delivering assembly 610). Alternatively or additionally, it will be appreciated that the configuration of the multi-electrode energy-delivering assembly 610 (e.g., monopolar, bipolar, or unipolar) affects/controls the ablation zone A. Furthermore, as may be appreciated, if the elongated shaft 624 from which the electrode tines 622 extend is polarized, an insulation layer or member 640 is provided between the elongated shaft 624 and the tubular electrode 630 to electrically isolate/insulate the electrically-conductive materials thereof from each other. The insulation member 640 may be a generally tubular member formed of insulative material (such as any of the insulative materials described above) which may be selected based on the material of the electrode tines 622 and/or the tubular electrode 630, and may extend the full longitudinal extent along which the elongate shaft 624 and the tubular electrode 630 are adjacent to each other.

In accordance with various principles of the present disclosure, an insulation sheath 660 may be provided over a proximal extent of the tubular electrode 630, such as to insulate the patient from energy delivered from an energy source coupled to a proximal end of the tubular electrode 630. Additionally or alternatively, the insulation sheath 660 may delimit an energy-delivery region 632 of the tubular electrode 630. The distance between the distal end 660d of the insulation sheath 660 and the distal end 630d of the tubular electrode 630 may be selected to generate an ablation zone A of the desired size/volume such as determined, in part, by the size of the electrode formed by the tubular electrode 630 (e.g., such as may be determined based on various characteristics of the target site to be treated by the multi-electrode energy-delivering assembly 610).

In some aspects, in accordance with various principles of the present disclosure, energy supplied to the various electrodes of a multi-electrode energy-delivering assembly with a multi-electrode needle such as illustrated in FIG. 6, FIG. 6A, FIG. 7, and FIG. 7A may be customizable. More particularly, in the example of an embodiment of a multi-electrode energy-delivering assembly 710 illustrated in FIG. 7 and FIG. 7A, each of the electrode tines 722 of the illustrated example of an embodiment of a multi-electrode needle 720 of the multi-electrode energy-delivering assembly 710 is electrically separated from one another, such as by being covered by an insulative material (e.g., any of the above-described insulative materials), with only the distal ends 720d thereof being uninsulated. As such, each of the electrode tines 722 may be activated individually, separate from the other electrode tines 722, such as to allow multi-polar operation of the multi-electrode energy-delivering assembly 710. Such individualized activation may allow customizable treatment zones between/among the electrode tines 722 and/or different customizable modes of operation and/or treatment of a target zone. Additionally or alternatively, the electrode tines 722 may be activated as a group, such as to allow monopolar operation of the multi-electrode energy-delivering assembly 710. Additionally or alternatively, the electrode tines 722 or 622 may be activated in two groups, such as to allow bipolar operation of the multi-electrode energy-delivering assembly 710 or 610. Similar to the multi-electrode energy-delivering assembly 610 illustrated in FIG. 6 and FIG. 6A, the multi-electrode energy-delivering assembly 710 illustrated in FIG. 7 and FIG. 7A has a tubular electrode 730 with an insulation sheath 760 may delimit an energy-delivery region 732 of the tubular electrode 730. For the sake of brevity, and without intent to limit, reference is made to the above description of FIG. 6 and FIG. 6A as generally applicable to the elements and features of FIG. 7 and FIG. 7A in this regard.

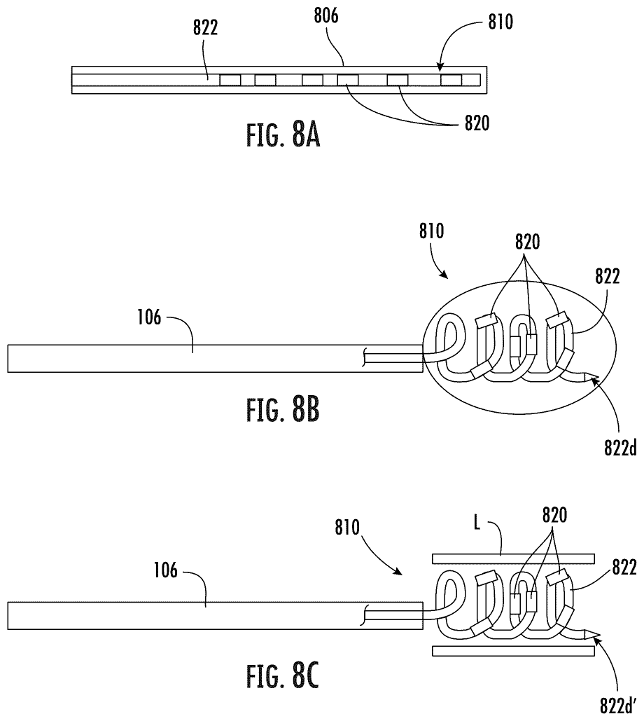

A further example of an embodiment of a multi-electrode energy-delivering assembly with multiple, separate electrodes is illustrated in FIG. 8A, FIG. 8B, and FIG. 8C. Similar to the multi-electrode energy-delivering assembly 210 illustrated in FIG. 2, the multi-electrode energy-delivering assembly 810 illustrated in FIG. 8A, FIG. 8B, and FIG. 8C has two or more electrodes which are mounted on a common element of the multi-electrode energy-delivering assembly 810 but which are spaced apart (e.g., axially) with respect to one another. However, whereas the multi-electrode energy-delivering assembly 210 illustrated in FIG. 2 is deployed for treatment in a generally elongated, linear configuration, the multi-electrode energy-delivering assembly 810 illustrated in FIG. 8A, FIG. 8B, and FIG. 8C may be delivered in a generally linear configuration, but need not have a generally linear configuration once deployed at a target site. As used herein, at least with respect to the example of an embodiment of a multi-electrode energy-delivering assembly 810, the term delivered (and other grammatical forms thereof) is generally intended to refer to the multi-electrode energy-delivering assembly 810 when positioned within a delivery device such as described above (such as within a delivery lumen/working channel of a delivery device 106 such as illustrated in FIG. 4). And, as used herein, at least with respect to the example of an embodiment of a multi-electrode energy-delivering assembly 810, the term deployed (and other grammatical forms thereof) is generally intended to refer to the multi-electrode energy-delivering assembly 810 once positioned outside a delivery device and ready to apply (and/or already applying) therapeutic treatment.

In a delivery configuration, such as illustrated in FIG. 8A, the multi-electrode energy-delivering assembly 810 may be delivered to a target site within a patient in a relatively compact configuration. As such, the multi-electrode energy-delivering assembly 810 may be delivered in a configuration which facilitates delivery within a protective sheath 806 (e.g., similar to the sheath 106 illustrated in FIG. 1) within the working channel of an endoscope or other delivery device which may be sized to navigate through anatomical passages, lumens, channels, etc., within a patient's body. For instance, the multi-electrode energy-delivering assembly 810 and protective sheath 806 may be delivered through a working channel of a currently available endoscope. Upon comparison of the delivery configuration of the multi-electrode energy-delivering assembly 810 illustrated in FIG. 8A, and the deployed configuration of the multi-electrode energy-delivering assembly 810 illustrated in FIG. 8B and FIG. 8C, the multi-electrode energy-delivering assembly 810 may transition from a generally elongated, compact delivery configuration to a generally expanded configuration. In a deployed configuration, such as illustrated in FIG. 8B and FIG. 8C, a multi-electrode energy-delivering assembly 810 formed in accordance with various principles of the present disclosure may be expanded from its compact delivery configuration to create a larger ablation zone than typically created by a linear multi-electrode energy-delivering assembly. Additionally or alternatively, a multi-electrode energy-delivering assembly 810 formed in accordance with various principles of the present disclosure may have a customizable shape or configuration to create a customized ablation zone, such as by forming at least a part of the multi-electrode energy-delivering assembly 810 from a shape memory material so that the multi-electrode energy-delivering assembly 810 expands to a pre-set configuration upon deployment.

In accordance with various principles of the present disclosure, two or more electrodes 820 are provided axially spaced along the length of a support element 822. Each electrode 820 may be independently wired or selectively wire to an anode and cathode and therefore conduct the electrical signal between the anode and cathode electrodes and fired simultaneously or multiplexed. Additionally or alternatively, the energy-delivering assembly 810 may be a single monopolar electrode. The support element 822 is flexible to shift from a delivery configuration to a deployed configuration such as described herein. In some aspects, the support element 822 is formed of a shape memory material such as any of those described herein, and/or a shape memory nylon or plastic. Additionally or alternatively, an actuation element may extend to the support element 822 to form the desired shape of the multi-electrode energy-delivering assembly 810 upon deployment from a delivery device 106, such as using a method similar to spring forming (e.g., a rotational and/or axial mechanism operatively coupled with the multi-electrode energy-delivering assembly 810 to form the desired shape, such as a coil, for deployment with respect to a treatment site). Additionally or alternatively, the electrodes 820 may be made of a shape memory alloy such as nitinol, spring tempered steel, etc.

In the expanded deployment configuration illustrated in FIG. 8B, the support element 822 may have a curved configuration, such as spiral, helical, corkscrew, etc., configuration. Moreover, the distal end 822d of the support element 822 may be sharp or pointed or otherwise configured to pierce and/or puncture tissue, such as to be inserted within tissue (e.g., a tumor) at a target site. A curved configuration of the support element 822 may allow the support element 822 to be rotated to effectively screw the support element 822 into tissue at the target site, such as to a selected insertion depth which may be assessed and monitored in any of a variety of manners known those of ordinary skill in the art.

In some aspects, such as illustrated in FIG. 8C, the multi-electrode energy-delivering assembly 810 may be expanded into an anatomical/body lumen L, such as a duct, intestine, blood vessel, etc. As may be appreciated, a preset expansion size may be selected based on the shape, size, configuration, and/or dimensions of the target site into which the multi-electrode energy-delivering assembly 810 is to be deployed. In some aspects, the distal end 822d′ of a support element 822 of a multi-electrode energy-delivering assembly 810 intended for deployment within a body lumen L may have an atraumatic, generally blunt shape, such as to avoid potential trauma to the target site upon being contacted by the distal end 822d of the support element 822.

The electrodes 820 of the multi-electrode energy-delivering assembly 810 illustrated in FIG. 8A, FIG. 8B, and FIG. 8C may be electrically-coupled with an energy source, such as described above with reference to the other examples of embodiments of energy-delivering assemblies of the present disclosure. The energy source may send multiplexed signals to the two or more electrodes 820 independently of one another, and the multi-electrode energy-delivering assembly 810 may be used in either a monopolar or bipolar mode.

It will be appreciated that any of the above-described multi-electrode probes may be used in a monopolar or bipolar manner. Moreover, it will be appreciated that provision of two, three, or more electrodes in accordance with various principles of the present disclosure described herein allow for various modifications and customizations of energy fields created by the energy-delivering assemblies described herein. It will be appreciated that the devices, systems, assemblies, and methods disclosed herein may be delivered endoscopically, or percutaneously, as well as use within other steerable luminal access devices. For instance, multi-electrode-energy delivering assemblies disclosed herein may be delivered in a configuration which is sufficiently compact to be delivered within a lumen of a delivery device which is inserted into and navigated within a patient to the target site at which the multi-electrode-energy delivering assembly is to be deployed. The configuration of the multiple (two, three, or more) electrodes and/or support structure therefor (e.g., one or more elements which may support the electrodes) of a multi-electrode energy-delivering assembly formed in accordance with various principles of the present disclosure allows for an energy field to be created which is larger and/or more customizable than achieved by a simple prior electrode/probe. For instance, an energy field formed by a multi-electrode energy-delivering assembly formed in accordance with various principles of the present disclosure may be able to produce larger and/or more customized energy field created than by two electrodes fixed on an elongate shaft, and/or may provide the ability to treat around a tumor mass or within a tumor based on need. Additionally or alternatively, a multi-electrode energy-delivering assembly formed in accordance with various principles of the present disclosure allows the shaping and sizing of the ablation zone based on the electrode(s) positioning and sizing in relation to each other along with the electrical waveform sent by the generator. Various of the components of the energy delivering assemblies described above may be formed of a shape memory material to allow formation of an electrode configuration suitable for treatment to be formed. The two, three, or more electrodes of any of the above-described multi-electrode energy-delivering assemblies may be coupled with an energy source outside the patient while the electrodes of the multi-electrode-energy delivering assembly are within the patient.

It will be appreciated that all structures, devices, systems, assemblies, and methods discussed herein are examples implemented in accordance with one or more principles of this disclosure, and are not the only way to implement these principles, and thus are not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, assemblies, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A multi-electrode energy-delivering assembly comprising:
one or more electrodes of a first polarity; and
a support structure coupling said one or more electrodes for delivery as an assembly within a patient;
wherein said multi-electrode energy delivering assembly has an elongated compact delivery configuration for delivery through a lumen of a delivery device.

2. The multi-electrode energy-delivering assembly of claim 1, wherein said one or more electrodes include two or more electrodes of the first polarity axially spaced from one another and extending in a linear configuration when in the elongated compact delivery configuration.

3. The multi-electrode energy-delivering assembly of claim 2, wherein said two or more electrodes are formed from an elongate electrically-conductive member and defined by an insulation member extending over and exposing portions of said elongate electrically-conductive member.

4. The multi-electrode energy-delivering assembly of claim 3, further comprising at least one electrode of a second polarity axially spaced from at least one of said two or more electrodes of the first polarity.

5. The multi-electrode energy-delivering assembly of claim 4, wherein said at least one electrode of a second polarity is formed from a tubular electrically-conductive member positioned over said insulation member.

6. The multi-electrode energy-delivering assembly of claim 5, further comprising at least one differentiating insulation member extending over a portion of said tubular electrically-conductive member to define two or more spaced apart electrodes of the second polarity.

7. The multi-electrode energy-delivering assembly of claim 2, wherein said two or more electrodes are positioned on a support element expandable from the elongated delivery configuration into an expanded deployed configuration when extended out of a delivery device to increase the volume of an electric field to be defined by the electrodes when said support element is in the deployed configuration compared with an electric field which would be defined by the electrodes when said support element is in the delivery configuration.

8. The multi-electrode energy-delivering assembly of claim 1, wherein said one or more electrodes comprise three or more electrodes movable with respect to one another.

9. The multi-electrode energy-delivering assembly of claim 8, wherein at least one of said three or more electrodes are configured to engage and remain engaged with tissue.

10. The multi-electrode energy-delivering assembly of claim 1, wherein said one or more electrodes of a first polarity comprises a fluid containing electrically-conductive particles injectable by the delivery device into a target site within a patient.

11. The multi-electrode energy-delivering assembly of claim 10, further comprising an additional electrode of a second polarity delivered to the target site with said fluid.

12. The multi-electrode energy-delivering assembly of claim 1, wherein said multi-electrode energy-delivering assembly is expandable upon deployment from a delivery device.

13. A multi-electrode energy-delivering treatment system comprising:
a multi-electrode energy-delivering assembly comprising:
one or more electrodes of a first polarity; and
a support structure coupling said one or more electrodes for delivery as an assembly within a patient;
a power connector configured to deliver energy to said one or more electrodes to generate an electric field among said one or more electrodes; and
a tubular delivery device having a lumen within which said multi-electrode energy-delivering assembly is deliverable to a target site within a patient;
wherein said multi-electrode energy delivering assembly is delivered within the lumen of said delivery device in an elongated compact delivery configuration.

14. The multi-electrode energy-delivering treatment system of claim 13, wherein said multi-electrode energy-delivering assembly is expandable upon deployment from the lumen of said delivery device.

15. The multi-electrode energy-delivering treatment system of claim 13, wherein said one or more electrodes of said multi-electrode energy-delivering assembly comprise two or more electrodes extending axially spaced apart from one another when in the lumen of said delivery device.

16. A method of applying therapeutic energy to a patient, said method comprising:
delivering, to a target site within a patient, a plurality of electrodes of a first polarity in an elongated delivery configuration within a lumen defined in a delivery device;
deploying the plurality of electrodes out of the lumen of the delivery device; and
delivering energy to the deployed plurality of electrodes to create an energy field between the plurality of electrodes.

17. The method of claim 16, further comprising spacing apart the electrodes of said plurality of electrodes from one another upon deployment thereof.

18. The method of claim 17, further comprising activating the plurality of electrodes independently of one another.

19. The method of claim 16, wherein said plurality of electrodes comprises a fluid containing a plurality of electrically-conductive particles, and delivering said plurality of electrodes further comprises injecting the fluid into the patient at the target site.

20. The method of claim 19, wherein the delivery device comprises an electrically-conductive material, said method further comprising delivering energy to the delivery device to generate an electric field between the electrically-conductive material of the delivery device and the electrically-conductive particles in the fluid.
